(19) United States
(12) Patent Application Publication
(10) Pub. No.: US 2022/0313724 A1
(43) Pub. Date: Oct. 6, 2022

(54) COMPOSITIONS FOR INTRATUMORAL ADMINISTRATION AND RELATED METHODS

(71) Applicants: HIGHLIGHT THERAPEUTICS, S.L., Valencia (ES); Fundación Para La Investigación Médica Aplicada, Pamplona (ES)

(72) Inventors: Ignacio MELERO BERMEJO, PAMPLONA (ES); Marisol QUINTERO ORTIZ, VALENCIA (ES); Maite ALVAREZ RODRIGUEZ, PAMPLONA (ES)

(21) Appl. No.: 17/744,634

(22) Filed: May 14, 2022

(30) Foreign Application Priority Data
May 17, 2021 (EP) 21 382 451.9

(51) Int. Cl. A61K 31/713; A61K 47/59; A61K 31/7084; A61K 39/395; A61P 35/00; A61K 9/00

(52) U.S. Cl. CPC A61K 31/713; A61K 47/59; A61K 31/7084; A61K 39/3955; A61P 35/00; A61K 9/0019

(57) ABSTRACT

The present invention relates to cancer treatment strategies comprising specific combinations of compounds or compositions that are administered intratumorally and can exert both local and distant anti-tumor effects. Preferred compounds and compositions to be combined for the intratumoral administration include a STING agonist and a TLR3 agonist. The disclosed combinations and related pharmaceutical compositions are additionally useful for inclusion in other cancer therapies such as radiotherapy or antibody-based immunotherapies.

### CROSS-REFERENCE TO RELATED APPLICATIONS

This application claims the benefit of European Patent Application EP 21 382 451.9, filed on May 17, 2021, the content of which is hereby incorporated by reference in its entirety.

### FIELD OF THE INVENTION

The present invention relates to the preparation and medical use of pharmaceutical formulations for intratumoral administration.

### BACKGROUND OF THE INVENTION

Intratumoral delivery of pharmaceutical compositions is known to provide certain advantages in terms of safety, efficacy and pharmacodynamics relating to injected compounds compared to other routes of administration. The intratumoral approach has been studied for the administration of various candidate drugs in mouse models for evaluating the local biological response both locally and in untreated or undetectable metastasis resulting from enhanced anti-tumor immunity or other responses attributable to the tumor environment. Such preclinical evidence can be translated into the development of clinical protocols useful for cancer immunotherapy (Aznar M et al., 2017; Marabelle A et al., 2017). If lesions located in the skin or close to the body surface are preferred for intratumoral delivery of pharmaceutical compounds, new technologies relating to interventional radiology and fluorescence-guided surgery for the purposes of image-guided drug delivery in internal organs can become more commonly available and also more efficient (Tselikas L et al., 2021; Pogue B et al., 2018).

Alternative strategies for intratumoral immunotherapy, which may also involve conventional standard-of-care approaches including radiotherapy or chemotherapy, are indicated as being feasible approaches in the clinic with promising results having been reported, for instance, through the use of unmethylated CpG oligonucleotides (Ribas A et al., 2018) and specific formulations comprising polyinosinic:polycytidylic acid [poly(I:C)] molecules such as BO-112 (Marquez-Rodas I et al., 2020; Aznar M et al., 2019) and poly-ICLC (Kyi C et al., 2018; Rodriguez-Ruiz M et al., 2018). Among the poly(I:C)-based compositions showing agonistic effects on double-stranded RNA intracellular sensors such as TLR3 (Toll-like Receptor 3) and/or MDA5, certain formulations referred to as BO-11X (WO 2017/085228; WO 2018/210439) comprise nanoplex particles that are formed by particular combinations of poly(I:C) molecules and polyethyleneimine molecules. The particles in these nanoplex formulations provide highly uniform sizes and other advantageous biophysical features that make such compositions suitable for clinical applications requiring a TLR3 agonist and compatible with requirements for production under GMP (Good Manufacturing Practices) conditions. In particular, the BO-112 formulation has been previously administered intratumorally in combination with the parenteral administration of monoclonal antibodies typically used in cancer immunotherapy as checkpoint inhibitors, including anti-PD-1 antibodies. Ongoing clinical trials evaluate BO-112 in combinations with radiotherapy and/or checkpoint inhibitors for the treatment of different cancers such as colorectal or gastric cancer with liver metastasis (NCT04508140), unresectable malignant melanoma (NCT04570332), PD-1/PD-L1 refractory liver cancer (NCT04777708), metastatic refractory Non-Small-Cell Lung Carcinoma (NCT05265650), or soft tissue sarcoma that can be removed by surgery (NCT04420975).

The cGAS/STING cytosolic DNA sensing pathway has also been shown to be critical for the induction of innate immunity against tumors (Motedayen Aval L et al., 2020; Flood B et al., 2019). Several compounds having different chemical features have been identified as STING agonists that show efficacy in cancer models only, and even in humans, which can be administered to a subject by a variety of different routes (including intratumorally). STING agonists can also be administered according to different drug combinations, and also represent tools for evaluating the effect on cell populations with particular importance in anti-tumor immunity modelling (WO 2015/077354; Corrales L et al., 2015; Ager C et al., 2017; Sivick K et al., 2018; Chelvanambi M et al., 2021).

TLR3 agonists, and in particular poly(I:C)-based compositions showing agonistic effects on double-stranded RNA sensors such as TLR3 or MDA5, and STING agonists can act on partially overlapping pathways. However, potential synergistic activities that may be observed by combining these agents have not been systematically evaluated or even compared with other drugs, in particular, with respect to demonstrating an improved efficacy for the intratumoral administration of pharmaceutical compositions.

### SUMMARY OF THE INVENTION

The present invention relates to novel uses and compositions for treating cancer involving TLR3 agonists and STING agonists suitable for intratumoral administration. The TLR3 agonist is preferably in the form of a composition comprising polyinosinic-polycytidylic acid [poly(I:C)] molecules, and more preferably comprising poly(I:C) molecules that are complexed in one or more particles with linear polyalkyleneimine molecules, in particular polyethyleneimine molecules that can be administered as an injectable, aqueous composition. Specific size and concentrations of the poly(I:C) molecules, together with polyethyleneimine molecules bearing certain physiochemical characteristics, allow providing TLR3 agonist-containing particles as compositions with convenient pharmaceutical features that produce a strong, localized effect in the injected tumor lesion that is not only compatible with, but also producing an amplified therapeutic effect of specific drugs known to well-suited for intratumoral injection. Such drugs preferably include one or more compounds that are defined in the literature as STING agonists, which in one preferred embodiment exist in the form of a small molecule, for example, a cyclic dinucleotide, a xanthenone, a flavonoid or a chemical derivative thereof, in both injected and non-injected tumors. Any of these STING agonists may be also directly included for use in known manufacturing processes that produce particles comprising polyinosinic-polycytidylic acid [poly(I:C)] molecules that are complexed in one or more particles with polyethyleneimine molecules. This combined intratumoral treatment approach may be advantageously combined with cancer treatment regimens that are administered by these same or alternative routes, including radiotherapy or antibody-based immunotherapies, (for instance, administration of an anti-PD-1 or anti-PD-L1 antibody), in particular when the cancer is metastatic and/or refractory or resistant to radiotherapy, chemotherapy or immunotherapy.

The compounds and compositions that are disclosed herein as TLR3 agonists and the STING agonists may be administered as two separate pharmaceutical compositions for intratumoral injection. Each such composition comprising either the TLR3 agonist or the STING agonist can be administered individually in the same tumor lesion(s) or in two or more different tumor lesions. Alternatively, the two pharmaceutical compositions may be administered together in a single co-formulation or composition comprising both the TLR3 agonist and the STING agonist in one or more tumor lesions. Such novel pharmaceutical compositions may be prepared just prior to administration by mixing the two compositions together, or can alternatively be produced by introducing a STING agonist during the process of manufacturing the TLR3 agonist composition, in particular when TLR3 agonist is a composition comprising poly(I:C) molecules that are complexed in one or more particles with polyethyleneimine molecules. In both cases, the ratio between the amount or the concentration of TLR3 agonist (as defined on the basis of particles or of poly(I:C) molecules contained in the composition) and the amount or the concentration of STING agonist in the composition may be defined according to the regimen and type or status of the cancer, considering also the overall status of the patient and the equipment needed for suitably guiding and performing the injection.

The location, number and frequency of each intratumoral injection of the pharmaceutical compositions disclosed herein may be defined according to the specific therapeutic needs and cancer status of the subject in need of such treatment, with injections that may be given simultaneously or concurrently (during the same medical act) or sequentially (in one or more distinct medical acts, separated by hours, days, or weeks), in two, three, or more cycles of treatment. Moreover, the combined, intratumoral administration of the TLR3 agonist-containing composition and the STING agonist-containing composition may be adapted to other cancer treatments and drug regimens that are administered (simultaneously or sequentially) using the same intratumoral route or alternatively, using other routes and means, including orally, subcutaneously, intradermally, intranasally, intravenously, intramuscularly, intrathecally, intranasally, intravesically, topically, and transdermally, which can improve the efficacy, safety, and/or clinical use of any of such drugs and treatments disclosed herein. Such drug and treatments to be combined with the described compositions and methods involving the intratumoral administration of the TLR3 agonist-containing composition and the STING agonist-containing composition can include surgery, radiotherapy, chemotherapy, toxin therapy, cancer vaccination, laser therapy, phototherapy, immunotherapy, cryotherapy or gene therapy. Preferably, the combination approach involves the administration of agents useful for cancer immunotherapies, for instance, an anti-PD-1 antibody or an anti-PD-L1 antibody (such as pembrolizumab or nivolumab).

The therapeutic approaches described herein comprising the intratumoral administration of the TLR3 agonist-containing composition and the STING agonist-containing composition may advantageously provide effects for treating cancers that are refractory or resistant to conventional treatments such as radiotherapy, chemotherapy or immunotherapy or other treatment strategies involving surgical excision, thermal or electrical ablation, cryotherapy, laser therapy, or phototherapy more effectively and/or in a more acceptable and beneficial manner for the patient. The therapeutic effects of intratumoral administration of the TLR3 agonist-containing composition and the STING agonist-containing composition may be defined as reducing the size of a tumor or inhibiting its growth, independent of injection, or a reduced resistance and/or increased sensitivity to another cancer therapy, for example, by overcoming escape mechanisms that may include mutations that alter specific genes, pathways, and/or response to drugs or endogenous compounds such as cytokines. Other therapeutic effects following intratumoral administration of the compounds or compositions described herein may comprise reducing malignancy and metastasis, reduction of the quantity or quality of medical interventions and/or the discomfort of a patient, increasing the amount of cell populations, for example, CD8+ T cells, cytokines including interferons, and/or biomarkers that are known to relate to an improved biological response in the treatment of cancer at various clinical stages, including Stage I, II, III, or IV.

The intratumoral administration of the TLR3 agonist-containing composition and the STING agonist-containing composition, or of a co-formulation of these compositions, may advantageously reduce the burden, persistence, metastasis, malignancy, and/or recurrence of a tumor, in particular if the tumor can be injected given its size, location, or other relevant features. The tumor can be identified to a wide variety of cancers, for example, melanoma, cervical cancer, liver cancer, breast cancer, ovarian cancer, pancreatic cancer, kidney cancer, prostate cancer, testicular cancer, urothelial carcinoma, bladder cancer, non-small/small cell lung cancer, colorectal adenocarcinoma, colorectal cancer, mesothelioma, gastrointestinal stromal tumors, biliary tract cancer, gastroesophageal carcinoma, myelodysplasia syndrome, transitional cell carcinoma, neuroblastoma, Wilms tumor, brain cancer, colon cancer, head/neck squamous cell carcinoma, hepatocellular carcinoma, and/or any other carcinoma or sarcoma. Exemplary, specific cancers are those refractory or resistant to radiotherapy, chemotherapy or immunotherapy, such as colorectal or gastric cancer, Non-Small-Cell Lung Carcinoma, liver cancer, soft tissue sarcoma, or malignant melanoma, and other solid tumors.

The two respective compositions described herein that are destined for intratumoral injection, comprising the TLR3 agonist-containing composition and/or the STING agonist-containing composition may be provided in one or more containers, individually or in combination, within a kit or a kit of parts. This kit or kit of parts may also include additional agents comprising one or more of diluents, adjuvants, excipients, syringes or other devices, and/or instructions for treating cancer using the composition(s) by intratumoral injection(s), optionally for use according to other cancer treatment protocols, in an individual presenting one or more specific cancer types, immunological or genetic features, and/or for use in one or more drug regimens.

Further embodiments, such as formulations, uses and methods according to the present invention, are disclosed in additional detail and exemplified in the Examples herein.

### BRIEF DESCRIPTION OF THE FIGURES

FIG. 1A shows a schematic representation of the approach for establishing the cancer model in mice is shown. An exemplary approach according to the invention for evaluating and comparing compounds and regimens useful for intratumoral cancer therapies. The animal model is established by subcutaneous injection of mouse cancer cells into the left and right flank of the mouse, for example, MC38 cells (murine colon carcinoma cells) or B16.OVA cells (murine melanoma cells), thus establishing a preclinical immuno-oncology model. When both the right and left tumors reach a significant tumor volume (for instance, 80-100 mm³), the intratumoral injection is performed mice in either or both tumor masses using a test compound (alone or in combinations) or using a vehicle as a control. The size of (non)injected tumors and mice percent of surviving mice is subsequently measured during the following 4-10 weeks. The dosing time course and regimen for intratumoral administration that is applied for each treatment over 24 days and involving BO-112 and/or the STING agonist DMXAA (FIG. 1B) or BO-112 and/or other compounds such as antibodies (anti-CD40) or CpG-based compounds (FIG. 1C), and related control is shown.

FIG. 2A shows an evaluation of the co-administration of BO-112 and DMXAA by intratumoral injection according to the invention. The single or combined administration of BO-112 and DMXAA is analyzed using the Standard Error of Mean (Mean±SEM) of tumor size volume (mm³) in treated or non-treated tumors in both MC38 and B16.OVA cancer models, as described in FIG. 1A, FIG. 1B, and FIG. 1C over a 4-week time period. This analysis is extended to assess animal survival in both models for up to 10 weeks (FIG. 2B). The data represents two independent experiments conducted over a total of three (for MC38 cells) or two (for B16.OVA cells) experiments using five to six mice per group. Two-Way ANOVA (for tumor size volume) or Log-rank test (for percent survival) were used to assess statistical significance. Statistically significant differences are displayed for comparisons of only BO-112 or DMXAA intratumoral administration with respect to intratumoral administration of BO-112 combined with DMXAA (**p<0.01, ***p<0.001, ****p<0.0001).

FIG. 3A and FIG. 3B show the effect of the co-administration of BO-112 together with an antibody or a CpG-based compound by intratumoral injection using the model and protocol shown and described in FIG. 1A, FIG. 1B, and FIG. 1C and FIG. 2A and FIG. 2B herein. The single or combined administration of BO-112 and anti-CD40 antibody or CpG class A oligonucleotide (ODN 1585) is evaluated in treated or non-treated tumors in the MC38 cancer models over the course of 4 weeks (FIG. 3A). The analysis is extended to evaluate animal survival in both models up to 10 weeks (FIG. 3B). Comparable results are generated using class B (ODN 1668) and class C (ODN 2395) CpG oligonucleotides. Data are representative of three independent experiments using five to six mice per group (mean±SEM). Statistical significance is determined in the same manner described in the experiments shown in FIG. 2A and FIG. 2B herein.

FIG. 4A and FIG. 4B show a comparison of the intratumoral treatment using BO-112 and the STING agonist DMXAA at the level of tumor size and presence of effector CD8 T cells in the tumor microenvironment. MC38 tumor-bearing mice received two doses of BO-112 and/or DMXAA following the dose regimen described in FIG. 1B and tumors were collected 24 hours following the last treatment. The four experimental group results are associated to the column using the indicated symbols and compared for the weight of tumors on day 11 (FIG. 4A), total number of CD8 T cells per gram of tumor (FIG. 4B), the number of gp70-positive CD8 T cells (FIG. 4C), and the ratio between CD8 and Tregs in the tumor (FIG. 4D). Data represents an experiment with six mice per group for flow cytometry (mean±SEM). The one-Way ANOVA test was used to assess statistical significance. Significant differences are displayed for comparisons of only BO-112 or DMXAA intratumoral administration with respect to intratumoral administration of BO-112 combined with DMXAA (*p<0.05, **p<0.01, ***p<0.001, ****p<0.0001).

FIG. 5A shows a schematic representation of the approach for establishing the cancer model using MC38 cells in mice. Comparison of the intratumoral treatment using BO-112 and the STING agonist DMXAA in the same or different tumors in a mice model bearing three tumor lesions. The dosing time course and regimen is followed for each treatment over a period of 24 days using BO-112 and/or the STING agonist DMXAA, with the same protocol described above in connection with FIG. 1B. The three treatment groups (untreated control, treated with two intratumoral injections in a single tumor lesion, or treated with two intratumoral injections in two distinct tumor lesions) are compared for average tumor growth (mean±SEM) over five weeks in relation to an untreated tumor lesion for all groups in either right or left flank tumors, treated or untreated depending the treatment group in the latter case (FIG. 5B). Data are representative of two independent experiments with six mice per group (Mean±SEM). Statistical significance is determined and shown similarly to the results depicted in FIG. 2A and FIG. 2B above.

FIG. 6A and FIG. 6B show an abscopal effect of intratumoral co-injection of BO-112 with DMXAA combined with systemic PD-1 blockade. B16.OVA tumor bearing mice were established and treated with intratumoral injections of BO-112 and DMXAA as described in FIG. 1A, FIG. 1B and FIG. 1C above. Mice received either the anti-PD-1 monoclonal antibody or control rat IgG on days 8, 10 and 12 post-tumor cell inoculation. The average tumor size (mean±SEM) is followed for treated and untreated tumors over 4 weeks (FIG. 6A). The percentage of survival is shown for the indicated four treatment groups over 10 weeks. Data represent an experiment using six mice per group (mean±SEM) (FIG. 6B). Statistical significance is determined and shown similarly to the results depicted in FIG. 2A and FIG. 2B.

### DETAILED DESCRIPTION OF THE INVENTION

The present invention relates to therapeutic, abscopal effects provided by poly(I:C) molecules that are complexed in one or more particles with polyethyleneimine molecules, which may be provided as an injectable, aqueous composition (as exemplified by BO-11X formulations) when administered by intratumoral injection in combination with a specific class of oncological drugs, namely STING agonists. This approach may be useful for achieving an improved clinical efficacy by means of other treatments that are not administered intratumorally, for example, conventional radiotherapy or cancer immunotherapies. Such improvements disclosed herein have been observed in animal models that showed drug synergism for treating non-directly injected tumor lesions. These findings can then be translated to eradicate distant, untreated tumor lesions in clinical practice by the intratumoral administration of pharmaceutical compositions already under clinical development, for instance, BO-112 (a specific BO-11X formulation) in addition to pharmaceutical compounds that contain a compound that acts as an agonist for human STING (in particular nucleotidic or non-nucleotidic STING agonists under clinical development). Such clinical regimens and protocols can include other cancer-specific interventions such as localized radiotherapy or parenteral administration of anti-PD-1-antibodies such as pembrolizumab or nivolumab.

The dose and regimen optimization strategies described herein may be defined on the basis of known properties and alternative formats of clinically relevant compounds. In the case of BO-11X formulations, the biophysical properties and poly(I:C) size and/or amount to be administered intratumorally may be evaluated on the basis according to WO 2017/085228 and WO 2018/210439, herein incorporated by reference. Moreover, WO 2020/104628, also herein incorporated by reference, and other public disclosures reporting clinical studies involving BO-112 formulations or its labelled derivatives (BO-11XL) may be exploited to additionally adjust the dosage of the pharmaceutical compositions, the regimen of administration, the selection of patients to be treated, any additional medical interventions (including surgery or other tumor ablation techniques), and the appropriate clinically useful criteria to assess the outcome of BO-112 intratumoral administration when combined with the intratumoral administration of the STING agonist-containing composition.

Several known STING agonists are currently in preclinical or clinical development, with a series of clinical trials performed for various cancer indications (Le Naour J et al., 2020b; Motedayen Aval L et al., 2020; Harrington K et al., 2018; Zandberg D et al., 2020). A composition comprising any of these STING agonists is well suited for combination with a TLR3 agonist-containing composition by intratumoral administration. Exemplary STING agonists include nucleotidic compounds based on cyclic nucleotides, in particular GSK-532, MK-2118, MK-1454, IMSA-101, ADU-S100 (also known ML-RR-52-CDA or MIW815), and their respective chemical derivatives such as E7766. Other examples of STING agonists include non-nucleotidic compounds based on xanthenone, flavonoids and other flavone acetic acid derivatives such as DMXAA (5,6-dimethylxanthenone-4-acetic acid; also known ASA404 or Vadimezan), BMS-986301, GSK-3745417, and their chemical derivatives. Intratumoral administration of STING agonists may be improved by making use of any of the drug delivery technologies that have been reported for this category of compounds, for instance, delivery by liposomes, polymers or hydrogels (Motedayen Aval L et al., 2020).

In one preferred embodiment, the BO-11X formulation (the exemplary TLR3 agonist-containing composition) and/or STING agonist-containing composition for intratumoral administration as described herein may be administered as a single, injectable formulation comprising BO-11X and the STING agonist in a combination. Such pharmaceutical formulation may be produced by, for instance, mixing the formulation comprising the STING agonist with a BO-11X formulation or by adapting BO-11X preparation by including the STING agonist in any step of the manufacturing process, being a specific formulation under the definition of BO-11Xm as described in WO 2018/210439. These combined formulations for intratumoral administration (which may be referred to as BO-11X-ITSA or, more specifically BO-112-ITSA) can be generated by using alternative combinations of STING agonist concentrations or amounts with respect to any of the features normally characterizing a BO-11X formulation such as the amount or percentage of poly(I:C) molecules in a given size range, for instance, between 400 and 5000 base pairs, having less than 400 base pairs, having more than 5000 base pairs or any other range described in WO 2017/085228 or WO 2018/210439, and/or the amount or concentration of the particles comprising poly(I:C) molecules that have a given size as defined by z-average, median (D50%), and/or mono-modal diameter in any range or percentage that is described in WO 2017/085228 and WO 2018/210439. Moreover, together with, or alternative to a STING agonist, the preparation of a BO-11Xm formulation for intratumoral administration may optionally include small interfering RNA, aptamers, antisense oligonucleotides, mRNA, or other molecules that are under preclinical validation or clinical development as oligonucleotide therapeutics in oncology (Xiong H et al., 2021).

The BO-11X-ITSA formulations described herein present biological and biophysical features comparable with features of earlier known BO-11X formulations, for example BO-112 formulations, but may provide significant improvements for the therapeutic management of cancer, in particular with respect to untreated tumor lesions, circulating cancer cells, (micro)metastasis and other damage produced by the persistence of cancer cells in the body, thus reducing tumor burden, persistence, metastasis, malignancy, and/or recurrence. These clinical effects may be evaluated not only at the level of standard criteria for evaluating cancer progression or invasiveness but also by evaluating anti-cancer immunological responses that are mediated by specific (sub-) populations of immune cells (such as antigen-specific CD8+ T cells, dendritic cells, T regulatory cells, T cells and/or NK cells). These properties may be evaluated by the analysis of data obtained by exposing cell preparations, tissues, organs, animals, organoids, human or clinical samples (e.g., biopsies, blood or plasma preparations) or other biological samples or extracts useful for cancer detection and potentially for defining which distinct biological targets and effects are a direct result of, or associated with, the biological activities that BO-11X-ITSA formulations exert on such cells (e.g., apoptosis, autophagy, activation, proliferation, or other) or such cells exert in human body (within the tumor, in lymph nodes, in blood and the like).

The biological effects of BO-11X-ITSA formulations may be detected and/or defined using a variety of physiological criteria, for instance, changes in the combined secretion of chemokines, cytokines, interferons or in the expression of specific cell surface receptor or transcription factors. These observations can be useful for validating the therapeutic use of BO-11X-ITSA formulations alone or in a combination therapy with other drugs or treatments such as radiotherapy, cancer immunotherapy, or tumor ablation. In particular, when compared to clinical or therapeutic effects in cancer models or patients that are treated (or can be a potential candidate of treatment) with only either a STING agonist or a BO-11X formulation in parallel or in comparable situations, may advantageously permit the identification of novel clinical uses, methods of treatment and drug regimens that may benefit from treatment by BO-11X-ITSA formulations, alone or in combination with other drugs, including anti-cancer drugs (e.g., standard-of-care or antibodies against cancer antigens), either during the course of, or prior to, the treatment. The technologies for image-guided, drug delivery in internal organs (Tselikas L et al., 2021; Pogue B et al., 2018) may be helpful for choosing and injecting the tumor lesions to be directly treated.

The amount or the concentration of TLR3 agonist (which can be defined on the basis of particles or of poly(I:C) molecules as described herein) and the amount or the concentration of the STING agonist within the separate pharmaceutical compositions destined for intratumoral administration (or within a BO-11X-ITSA formulation) can be determined on the basis of the published data generated during the preclinical and clinical studies involving either agent when also administered intratumorally. For instance, a STING agonist may be administered by means of a single injection at a concentration comprised between 0.01 and 500 μg/kg, including any intermediate value such as 0.05, 0.1, 0.5, 1.0, 2, 5, 10, 20, 50, 100, or 250 μg/kg. In the case of a TLR3 agonist, and in particular the described particles comprising poly(I:C) molecules complexed with polyethyleneimines, the dose to be administered by means of a single injection can be determined on the amount of poly(I:C) molecules contained therein, for instance, between 0.01 and 5000 mg, including any intermediate value, for example, 0.05, 0.1, 0.5, 1.0, 2, 5, 10, 20, 50, 100, 250, or 500 mg, but preferably, from about 0.5 mg to about 2.5 mg of poly(I:C) molecules.

Alternatively, the amount of either the TLR3 agonist or the STING agonist can be defined as the total amount to be injected in two or more injections during a single or multiple cycles or treatment (e.g., on a daily, weekly, or monthly basis). The ratio between the amount of the two agents may be adapted during further studies in which the concentration of either agent is reduced to validate the minimal amount of the compound that is required to obtain the synergistic, therapeutic effect on injected and non-injected tumor lesions by means of minimal number of intratumoral injections, while limiting undesirable effects or poor compliancy to the treatment. Equally, the volume of BO-11X-ITSA formulation (or separate BO-11X and STING agonist formulations) that is injected can be adjusted accordingly (from 0.01 ml, 0.1 ml, 0.5 ml, 1 ml, 5 ml or more per injection) to provide the agents at lower or higher local concentrations, that may be further adapted during each cycle of treatment as needed.

The present invention additionally provides methods of treatment and other medical regimens that may benefit from the intratumoral administration of a TLR3 agonist-containing composition, such as a BO-11X formulation and in particular a BO-112 formulation, in combination with the intratumoral administration of a STING agonist-containing composition, administering each such composition separately, or, alternatively, in a single composition or formulation, such as a BO-11X-ITSA formulation and in particular a BO-112-ITSA formulation. The methods of treatment and medical regimens as described herein may be suitable for a given cancer (i.e. a specific type of cancer according its pathophysiology, metastatic properties, location, stage, etc.), populations of candidate patients (i.e. as defined by prior treatments, ongoing standard-of-care or other treatment, immunological profile, altered gene copy number, or genetic mutations or activation of relevant genes), and/or combination with other drugs (such as checkpoint inhibitors, antibodies targeting cancer antigens, cancer vaccines, non-human antigens, or adoptive cell therapies). In addition, the comparison of effects exerted by a BO-11X-ITSA such as a BO-112-ITSA formulation, with such other therapeutic compounds in clinically relevant models would advantageously permit the identification of the patients, or sub-populations of patients, the immunological profile, and/or the cancer stage or type (as those generally defined in cancer) which may positively respond to a treatment based on a BO-11X-ITSA formulations.

These alternative, novel therapeutic drug regimens may involve the administration of a BO-11X-ITSA formulation (or separate BO-11X and STING agonist formulations) by intratumoral injection at specific pathological altered locations (such as cancer lesions) within a tissue, an organ, and/or skin. The co-administration of the separate formulations, or of the BO-11X-ITSA co-formulation, may be performed in the same tumor lesion or in two or more tumor lesions, as determined by a clinician, for example, in accordance with clinical parameters such as a suitable clinical response defined by the analysis of biomarkers, gene expression signatures, cancer antigens, immune cells, or clinical criteria (e.g., tumor burden, stage of the tumor, amount of metastasis, and/or tumor recurrence). Moreover, the relevant institutions responsible for the evaluation and/or control of clinical therapeutics (such as FDA, EMA, WHO and ICLIO) provide the relevant criteria for evaluating a response to a drug treatment in cancer patients, in general or for specific drugs (e.g. immunotherapies), specific technologies (e.g. PET and imaging), specific biomarkers, and/or specific types of cancers (Liberini V et al, 2021; Lang D et al 2020; van de Donk P et al., 2020).

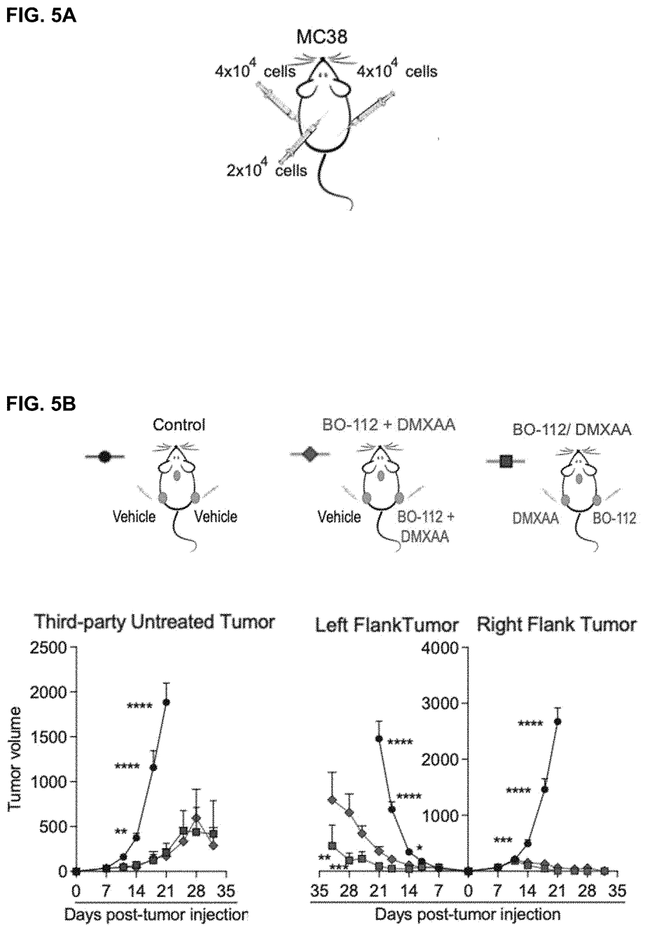

Patients that are resistant or insensitive to an anti-cancer agent (in general or only at level of tumor not directly treated with such agent) may be preferably treated by one or more intratumoral injections of a BO-11X-ITSA formulation, or alternatively, a separate BO-11X and STING agonist formulations. In one embodiment, the combination approach may involve the administration of one or more anti-PD-1 and anti-PD-L1 antibodies (anti-PD-1/PD-L1) whose efficacy as a cancer immunotherapeutic agent may be improved (or simply made possible) by modifying an existing regimen involving the use of these drugs (involving regular intravenous injections of the antibody) by including an intratumoral administration of a BO-11X-ITSA formulation (or separate BO-11X and STING agonist formulations) into one or more tumor lesions, one or multiple times, for instance, according to the data shown in FIG. 5A and FIG. 5B herein. This regimen may be initiated either before the beginning of a standard anti-PD-1/PD-L1 clinical protocol or concomitantly with the anti-PD-1/PD-L1 protocol, for instance, by administering anti-PD-1/PD-L1 either every 4, every 3, or every 2 weeks. The intratumoral injections of the BO-11X-ITSA formulation (or separate BO-11X and STING agonist formulations) can be performed every week (or daily) when the anti-PD-1/PD-L1 antibody is also administered.

Alternatively, the drug regimen may include specific weeks in which only either intratumoral injections of the BO-11X-ITSA formulation (or separate BO-11X and STING agonist formulations) or anti-PD-1/PD-L1 administration (e.g. by intravenous injection) is performed. This drug regimen may also include a maintenance cycle in which either the intratumoral injection(s) or other drug administration are alternatively or concomitantly administered over a number of weeks, for instance, 2, 4, 6, 8, 10, 12, or more weeks, and such cycle is repeated 2, 3, 4 or more times. The drug regimen may further comprise an intratumoral administration of a TLR3 agonist and/or a STING agonist at least two, three, four, five, six, seven, eight, nine or ten times in each cycle. The intratumoral administration of the BO-11X-ITSA formulation (or of respective BO-11X and STING agonist formulations) in each cycle may be separated by a predefined number of hours, for example, 1, 3, 6, 12, 18, 24, 36 or more hours, or by a predefined number of days, for instance, 2, 3, 5, 7, 10 or more days, over a period of 1, 2, 3, 4 or more weeks.

A cancer patient may be identified as a suitable candidate for intratumoral injections of BO-11X-ITSA formulation (or separate BO-11X and STING agonist formulations) according to the present invention, as a first line, second line, or later line of treatment, since presenting the appropriate combination of clinical parameters and/or biomarkers and/or not responding to standard-of-care cancer therapies (such as radiotherapy or chemotherapy) or drugs that target to specific cancer antigens, immune checkpoints, cells, or biological pathways. These drugs may be of a different chemical nature, for example, the drug can be a small molecule (e.g. inhibitors of kinases or other enzymes), peptides (such as cancer vaccines), nucleotides (e.g. oligonucleotides, aptamers, mRNA, small interfering RNA), antibodies, (e.g. directed against PD-1, PD-L1, CTLA4, or CD20), cell-based products (e.g. adoptive cell transfer as in adoptive T cell therapy and adoptive immunotherapies in general), or nucleic acids (e.g. targeting TLR9 or other receptor, DNA vectors encoding specific cancer antigens or therapeutic proteins). With respect to antibodies directed to cancer antigens, exemplary antigens are those approved (or under validation and review) by regulatory authorities including the EMA (in Europe) or the FDA (in the USA), in addition to targeting cell surface proteins, in particular for treating solid tumors, and characterized by a therapeutic activity and/or a clinical use that may be improved by a combined administration with a pharmaceutical composition having immunostimulatory activities, including one or more intratumoral injections of a BO-11X-ITSA formulation (or separate BO-11X and STING agonist formulations). Exemplary antigens include PDGFRα, PDGFRα), Her2, EGFR, VEGFR2, EpCAM, CD40, and CD25 (Zahavi D and Weiner L, 2020).

The present invention further relates to uses and methods applicable to the pharmaceutical compositions comprising nanoplex particles that are disclosed in WO 2017/085228 and WO 2018/210439 with respect to the composition components, manufacturing of the composition, biophysical features and biological activities of the composition, and other criteria that specifically apply, or are compatible with, intratumoral administration and related regulatory requirements, such as GMP (Good Manufacturing Practices) guidelines, and international standards (in particular, ISO 22412 and ISO 13099). In addition, the BO-11X-ITSA formulation (or separate BO-11X and STING agonist formulations) for intratumoral injections may contain nanoplex particles in which Poly(I:C) molecules and/or the polyethyleneimine (PEI) molecules is fluorescently labelled as disclosed in WO 2020/104628, for example, using Cy5 as a preferred fluorophore for labelling the polyethyleneimine component of the nanoplex particles and Rhodamine as a preferred fluorophore for labelling poly(I:C) molecules comprised in the nanoplex particles.

Similar to the previously disclosed BO-11X, BO-11Xm, and BO-11XL formulations, the BO-11X-ITSA formulation (or separate BO-11X and STING agonist formulations) useful for one or more intratumoral injection may further comprise at least one pharmaceutically acceptable carrier, organic solvent, excipient and/or adjuvant, either included in the particles themselves or added to the aqueous composition, for example, glucose added at a concentration of between 1 and 10% (weight/volume). Other, alternative components suitable for the BO-11X-ITSA formulation (or separate BO-11X and STING agonist formulations) are described in WO 2017/085228, WO 2018/210439, WO 2020/104628, or in the reference literature such as in Remington's Pharmaceutical Sciences (edited by Adeboye Adejare; 23^{rd} edition, 2020).

A BO-11X-ITSA formulation (or separate BO-11X and STING agonist formulations) may conveniently be presented in a unit dosage form and may be prepared by any of the methods known in the art of pharmacy for intratumoral administration. Such methods generally include bringing the therapeutic agents into association with a carrier, which constitutes one or more accessory ingredients, and may vary according to the particular method of treatment, the particular dosage form, and the mode of administration. Numerous factors may modify the physiological action of BO-11X-ITSA formulation (or separate BO-11X and STING agonist formulations) such as body weight, gender, diet, time of administration, rate of excretion, condition of the subject, drug combinations, genetic disposition and reaction sensitivities and can be taken into account by those skilled in the art. Administration of the formulations can be carried out continuously or in one or more discrete doses within the maximum tolerated dose, adapting, as needed, any other drug or standard-of-care treatment.

The methods of treatment and uses according to the present invention relate to treating or preventing a cell growth disorder characterized by abnormal growth of human or animal cells, for instance, due to cancer (that is, involving tumorigenic transformation, metastasis, and/or any tumorigenic compound). Exemplary models for evaluating the methods or treatment and uses of the present invention are described in WO 2017/085228, WO 2018/210439, WO 2020/104628, and in the Examples described below. Preferably, the methods of treatment and uses of the present invention are intended for inducing (directly or indirectly) the death of one or more tumor cells or suppressing growth of the one or more tumor cells, and further including treating, reducing, ameliorating, or preventing cancer growth (in particular to obtain permanent growth arrest or senescence of the tumor and cancer cells), survival, metastasis, epithelial-mesenchymal transition, immunologic escape or recurrence. In some embodiments, the BO-11X-ITSA formulation (or separate BO-11X and STING agonist formulations) is used to treat cancers at various stages (e.g., Stage I, or Stage II, or Stage III, or Stage IV). By way of non-limiting example, using the overall stage grouping, Stage I cancers are localized to one part of the body; Stage II cancers are locally advanced, as are Stage III cancers. Whether a cancer is designated as Stage II or Stage III usually depends on the specific type of cancer. In one non-limiting example, Hodgkin's disease, Stage II indicates affected lymph nodes on only one side of the diaphragm, whereas Stage III indicates affected lymph nodes above and below the diaphragm. The specific criteria for Stages II and III therefore differ according to diagnosis. Stage IV cancers have often metastasized or spread to other organs or throughout the body. Alternatively, the methods of treatment and drug regimens according to the present invention are used for adjuvant therapy, i.e., a treatment that is given in addition to the primary, main or initial treatment. By way of non-limiting example, adjuvant therapy may be an additional treatment usually given after surgery where all detectable disease (in the form of tumors or other lesions, for instance) has been removed, but where there remains a statistical risk of relapse due to occult disease. In some embodiments, the agents described herein are used as an adjuvant therapy in the treatment of a cancer.

In present invention, the cancer treated by the disclosed compositions, combinations, drug regimens, and related methods of administration is one or more of following cancers when injectable: basal cell carcinoma, biliary tract cancer; bladder cancer; bone cancer; brain and central nervous system cancer; breast cancer; cancer of the peritoneum; choriocarcinoma; connective tissue cancer; cancer of the digestive system (including esophageal, stomach, colon, rectal or other gastrointestinal cancer); eye cancer; cancer of the head and neck; glioblastoma; hepatic carcinoma; hepatoma; intra-epithelial neoplasm; kidney, adrenal, or renal cancer; leukemia; liver cancer; lung cancer (e.g. small-cell lung cancer, non-small cell lung cancer, lung adenocarcinoma, and lung squamous carcinoma); melanoma; renal cell carcinoma; myeloma; neuroblastoma; oral cavity cancer (lip, larynx, tongue, mouth, and pharynx); pancreatic cancer; prostate cancer; retinoblastoma; rhabdomyosarcoma; cancer of the respiratory system; salivary gland carcinoma; skin cancer; squamous cell cancer; testicular cancer; thyroid cancer; uterine, endometrial, cervical, vulval, ovarian or other gynecological cancer; cancer of the urinary system; lymphoma including B-cell lymphoma, Hodgkin's and non-Hodgkin's lymphoma (NHL; including specific types such as low grade/follicular, small lymphocytic, intermediate grade/follicular, intermediate grade diffuse, high grade immunoblastic, high grade lymphoblastic, high grade small non-cleaved cell, or bulky disease NHL), mantle cell and AIDS-related lymphoma; chronic lymphocytic leukemia; acute lymphoblastic leukemia; Hairy cell leukemia; chronic myeloblastic leukemia; as well as other carcinomas and sarcomas; post-transplant lymphoproliferative disorder (PTLD), abnormal vascular proliferation associated with phakomatoses or edema (such as those that associated with brain tumors). Additional types and definition can be found in National Cancer Institute website as in the webpage dedicated to cancer types at https://www.cancer.gov/types.

In some embodiments, the cancer is a biliary tract cancer. In further embodiments, the biliary tract cancer is selected from pancreatic cancer, gallbladder cancer, bile duct cancer, and cancer of the ampulla of Vater. In some embodiments, the cancer is liver cancer. In some embodiments, the cancer is colon cancer. In some embodiments, the biliary tract cancer is cholangiocarcinoma and/or an adenocarcinoma. Alternatively, the cancer may be listed among the list of rare diseases, being defined according to the criteria of incidence and/or prevalence as defined in Europe or USA and indicated in the regularly updated lists made available in the website of organisations such as the International Rare Cancers Initiative (https://project.eortc.org/irci/).

More preferably, a BO-11X-ITSA formulation (or separate BO-11X and STING agonist formulations) according to the invention are used in methods of treatment for treating solid tumors, such as carcinomas, gliomas, melanomas, or sarcomas. The BO-11X-ITSA formulation (or separate BO-11X and STING agonist formulations) described herein are administered directly within or at a location proximal to the tumor, for example, at the margin of the tumor mass, in the surrounding epithelial cells, lymphatic and/or blood vessels including by intratumoral or peritumoral injection. The cancer may be a dormant tumor, which may result from the metastasis of a cancer. The dormant tumor may also be left over from surgical resection or removal of a tumor. The cancer recurrence may, for example, be tumor regrowth, a lung metastasis, or a liver metastasis, wherein the methods of treatments and uses of the invention may reduce or block metastasis in distant sites that tumor would have caused.

Additionally, macroscopic examination of organs and skin and microscopic, pathological analysis in either immune-deficient fully immune competent animal models may further indicate the efficacy of the methods and uses according to the present invention. The quantitative data that are generated in similar studies can be compared among the different experimental groups by using the appropriate statistical tests, with and without corrections for multiple testing, at the scope to evaluate which therapeutic (in particular anti-tumor) effects are provided by the administration of a BO-11X-ITSA formulation (or separate BO-11X and STING agonist formulations), alone or in combination with an additional drug. Moreover, the methods of treatment and drug regimens according to the present invention may improve the effect of a BO-11X-ITSA formulation (or separate BO-11X and STING agonist formulations) in inducing local or systemic immunity by promoting immune cell memory allowing its use as single therapeutic agent, or in combination with immunomodulatory compounds, tumor-targeting agents, oncolytic viruses, adoptive cell transfer and other cell-based therapies, DNA- or peptide-based vaccines, or inhibitors of immunosuppressive or other cancer-related metabolic pathway.

Furthermore, the disclosed compositions can be administered for supporting vaccines, cytokines, antigens, antibodies, chemical compounds, and other compounds having immunomodulatory activities for treating or preventing cancers (solid or not) or infection, for instance as adjuvant and/or for rescuing patients poorly responding or resistant to a drug, including agents for cancer immunotherapy, for altering cell metabolism and/or functions (preferably, in immune and/or cancer cells), for modulating DNA expression, replication and/or repair (including drugs that target epigenetic mechanisms), or for standard-of-care therapies (such as chemo- or radiotherapy, or vaccine-based therapies involving cancer or viral antigens). Such additional agent (in the form of protein or corresponding mRNA) that is co-administered (subsequently, in any order) with a BO-11X-ITSA formulation (or separate BO-11X and STING agonist formulations) may improve a method of treatment with respect to the bioavailability, efficacy, pharmacokinetic and/or pharmacodynamic profiles, stability, metabolization, or other pharmaceutical property when treatment with a BO-11X-ITSA formulation (or separate BO-11X and STING agonist formulations) or another compound of pharmaceutical interest is administered alone.

The methods of treatment and regimens of the present invention can also involve the administration of an immune-modulating agent that is preferably an antibody including a monoclonal antibody and other antibody formats, or any other pharmaceutically available agent that binds a cell surface protein that control immune response, thus acting as a checkpoint inhibitor (CPI), which can block, reduce and/or inhibit PD-1 and PD-L1 or PD-L2 and/or the binding of PD-1 with PD-L1 or PD-L2. Alternatively, the CPI can block, reduce and/or inhibit the activity of other immune checkpoint molecules such as LAG3, ICOS, CD137, CTLA-4, AP2M1, LAG3, OX-40, CD80, CD86, SHP-2, and/or PPP2R5A. As a further alternative, the CPI increases and/or stimulates CD137 (4-1BB) and/or the binding of CD137 (4-1BB) with one or more of a 4-1BB ligand and TRAF2. Other examples of a second therapeutic agent having immunomodulating properties include radiotherapy, chemotherapy CAR-T cells, cancer antigen vaccines, or agents that target regulatory T cells, metabolic enzymes, DNA repair and/or replication. The present invention also provides combining a BO-11X-ITSA formulation (or separate BO-11X and STING agonist formulations) with an immunomodulatory agent and/or with one or more common cancer treatment regimens (e.g., FOLFOX, FOLFIRI, radiation, photodynamic therapy or an antiproliferative agent such as doxorubicin, daunorubicin, mitomycin, actinomycin D, bleomycin, cisplatin, VP16, enedyine, vincristine, vinblastine, carmustine, and the like.

Exemplary cancer indications wherein the methods of treatment and drug regimens according to the present invention can be pursued by appropriately combining the administration of an agent inhibiting PD-1 (such an anti-PD-1 antibody) with or without a standard-of care treatment (for instance, radiotherapy, as adjuvant), include, but are not limited to, melanoma, triple negative breast cancer, sarcoma, head-and-neck cancer, colorectal cancer, bladder cancer, renal cell carcinoma, liver metastasis, gastric cancer, prostate cancer, and hepatocellular carcinoma. Such indications may be also treated by administering a BO-11X-ITSA formulation (or separate BO-11X and STING agonist formulations) with an anti-PD-1, anti-PD-L1, an anti-CTLA4, or an anti-OX-40 antibody using an optimized drug regimen design. In some embodiments, the immune-modulating agent targets one or more of PD-1, PD-L1, and PD-L2. Preferably, the immune-modulating agent is a PD-1 inhibitor. In some embodiments, the immune-modulating agent is an antibody specific for one or more of PD-1, PD-L1, and PD-L2. Such immune-modulating agent is an antibody, including the non-limiting examples of nivolumab (ONO-4538/BMS-936558, MDX1106, OPDIVO), pembrolizumab (KEYTRUDA), pidilizumab (CT-011), MK-3475, BMS 936559, MPDL3280A. For example, the BO-11X-ITSA formulation (or separate BO-11X and STING agonist formulations) is combined with one or more of MPDL3280A (optionally with vemurafenib) and MEDI4736 (optionally with one or more of dabrafenib and trametinib) for the treatment of melanoma.

More particularly, the BO-11X-ITSA formulation (or separate BO-11X and STING agonist formulations) disclosed herein may be used for obtaining a synergistic therapeutic effect when administered with the second therapeutic agent, including reducing the regular dosage and/or frequency of administration of the second therapeutic agent (thus potentially reducing the need for additional medical intervention, an undesirable resistance to drugs, and/or a patient's overall discomfort). Moreover, the BO-11X-ITSA formulation (or separate BO-11X and STING agonist formulations) according to the invention, when administered with the second therapeutic agent, may allow treating patients that are resistant, insensitive, or presenting a poor clinical response to the second therapeutic agent, by overcoming any specific tumor resistance or escape mechanism (including mutations that alter specific genes, pathways, and/or response to drugs or endogenous compounds such as cytokines). Thus, the presently disclosed BO-11X-ITSA formulation (or separate BO-11X and STING agonist formulations) can be used in the form of a drug-rescuing or drug-sensitizing combination treatment, preferably for the treatment of cancer. Such methods of treatment may involve administering the BO-11X-ITSA formulation (or separate BO-11X and STING agonist formulations) by intratumoral or peritumoral injection (within the tumor, at the margin of the tumor mass, in the surrounding epithelial cells, lymphatic or blood vessels). Alternatively, other means may allow administering the BO-11X-ITSA formulation (or separate BO-11X and STING agonist formulations) directly within or in the proximity of cancer cells or organ comprising the cancer cells, and the systemic administration of an immunostimulatory agent.

The present invention also provides methods of treatment and drug regimens involving the administration of a BO-11X-ITSA formulation (or separate BO-11X and STING agonist formulations) for increasing an immune response against a pathogen or other undesirable biological agent, and in particular for enhancing an anti-tumor immune response, potentially acting itself as an immune-modulating agent. Such an effect may be monitored by measuring a tumor-related immune response at the tumor site and tumor microenvironment (or in the bloodstream, other biological fluids, and tissues) at the level of relevant cell types or subpopulations (e.g., dendritic cells, T regulatory cells, T cells and/or NK cells) and/or of immunological biomarkers (e.g., chemokines, growth factors, cytokines, and their receptors). In particular, when the BO-11X-ITSA formulation (or separate BO-11X and STING agonist formulations) is clinically administered by intratumoral injection, apoptosis and/or necrosis is observed in the tumor, thus potentially promoting the presentation of tumor antigens to resident dendritic cells. A signaling cascade may lead also to the recruitment of immune cells, in particular CD4+ and CD8+ T cells into the tumor mass promoting an immune effect against the tumor, contributing to the cytotoxic effect of BO-11X-ITSA formulation (or separate BO-11X and STING agonist formulations). Otherwise, as described and shown in the Examples herein, the administration of the disclosed BO-11X and STING agonist formulations may induce changes in the absolute value and/or in ratio of specific immune cell populations within tumors, lymph nodes, or both.

### EXAMPLES

### Example 1: Effects of Intratumoral Co-Injection of the Poly(I:C)-Based Formulation BO-112 with a Reference STING Agonist or Other Compounds in Animal Models

Materials & Methods

Cancer cell lines. B16.OVA mouse melanoma cells and MC38 mouse colon carcinoma cell lines were kindly gifted by Dr. Lieping Chen (Yale University, New Heaven, USA) and Dr. Karl E. Hellstrom (Univ. Washington, Seattle, USA) respectively, but are available also from other members of the research community. B16.OVA cells were derived from B16 cells by transduction with a cDNA encoding the ovalbumin gene, as described (Linardakis E et al., 2002). B16.OVA cells were derived from Grade III colon adenocarcinoma (Corbett T et al., 1975). Cells were grown in RPMI 1640 media supplemented with GlutaMAX™ (Gibco), 10% heat-inactivated FBS, 50 μM 2-mercaptoethanol, 100 U/mL penicillin, and 100 μg/mL streptomycin at 37° C. with 5% CO₂ (complete media). B16.OVA tumor cells were grown in complete media supplemented with 400 μg/mL Geneticin (Gibco). Cells were collected for tumor studies when reaching exponential growth during that week of culture.

Tested compounds. BO-112 was produced in accordance with WO 2017/085228 and WO 2018/210439. DMXAA, CpG ODN 1585, CpG ODN 1668, and CpG ODN 2395 were purchased from Invivogen (Toulouse France). Agonistic anti-CD40 antibody and rat IgG were purchased from BioXcell (Lebanon, N.H., USA).

Mouse tumor models. C57BL/6 mice were purchased from Harlan Laboratories (Barcelona, Spain). Female mice were used at 8-12 weeks of age and housed under specific pathogen-free conditions. The abscopal effect was studied by injecting 4×10⁵ MC38 or B16.OVA cells subcutaneously (s.c.) into the right flank of C57BL/6 mice, whereas the left flank received an injection (s.c.) of 2×10⁵ (MC38) or 1.25×10⁵ (B16.OVA) cancer cells. When right tumors reached a tumor volume of 80-100 mm³ (approximately one week after tumor inoculation), mice were randomized into different treatment groups.

Evaluation of compounds. Depending on the experiment, right tumors were injected intratumorally (i.t.) with one or more of the following compounds: BO-112 (50 μg), DMXAA as reference STING agonist (100 μg), TLR9 ligand/Class A CpG ODN 1585 (50 μg), TLR9 ligand/Class B CpG ODN 1668 (50 μg), TLR9 ligand/Class C CpG ODN 2395 (50 μg), agonistic anti-CD40 antibody (30 μg). Control mice received intratumoral injections of PBS containing 5% glucose and/or 5% DMSO (vehicle) as indicated. Treated and untreated tumors were measured twice a week with calipers and the volume was calculated (length×width²/2). Additionally, mice were monitored for survival and euthanized when any tumor size reached a diameter of 15 mm or mice displayed signs of distress. The spatial requirements for co-injections of BO-112 and DMXAA (i.t.) was evaluated by injecting (s.c.) 4×10⁵ MC38 tumor cells into the left and right flanks, and 2×10⁵ tumor cells in the upper dorsal region. When right and left tumors reached a diameter of 80-100 mm, mice were randomized to three groups. The control group received injections (i.t.) of 5% glucose and 5% DMSO in PBS to both right and left tumors, a BO-112+DMXAA group received BO-112 and DMXAA (i.t.) injections in the right tumor, and BO-112/DMXAA group received BO-112 (i.t.) injections in the right tumor and DMXAA (i.t.) in the left tumor. The control, untreated third-party tumor was untreated in all groups.

Flow cytometry. Organs were mechanically disrupted, and single-cell suspensions were generated as previously described (Aznar M et al., 2019). The distribution of immune cells in tumor and tumor lymph nodes was evaluated by flow cytometry. Single-cell suspensions were stained with immune cell marker-specific monoclonal antibodies as previously described (Alvarez M et al. 2019; Alvarez M. et al. 2020). CD8 T-cell compartment (CD45+CD19−TCRβ+NK1.1−CD8+CD4−) was analyzed for antigen specificity (gp70 pentamer). The Foxp3/TF staining buffer kit (eBioscience, San Diego, Calif., USA) was used according to manufacturer's instructions for intracellular staining. CD4 T cells and Tregs were defined as CD45⁺CD19⁻ TCRβ⁺NK1.1⁻CD8⁻CD4⁺Foxp3⁻and CD45⁺CD19⁻ TCRβ⁺NK1.1⁻CD8⁻CD4⁺CD25⁺Foxp3⁺, respectively. The monoclonal antibodies against the following antigens were used: CD11b (clone M1/70, BioLegend), CD11c (clone N418, BioLegend), CD19 (clone 6D5, BioLegend), CD274 (PD-L1, clone 10F.9G2, BioLegend), CD4 (clone GK1.5, BD Bioscience), CD44 (clone IM7), BioLegend), CD45 (clone 30.F11), BioLegend), CD62L (clone MEL-14, BioLegend), CD8 (clone 53-6.7, BD Bioscience), CD80 (clone 16-10A1, BioLegend), CD86 (clone GL-1, BioLegend), Foxp3 (clone MF-14, BioLegend), MHCII (clone M5/114.15.2, Thermo Fisher Scientific), TCRb (clone H57-597, BioLegend). Stained cells were analyzed with Cytoflex LX (Beckmann Coulter, Indianapolis, Ind., USA). Fluorescence minus one (FMO) or biological comparison controls were used for cell analysis. FlowJo software (TreeStar) was used for data analysis.

Multiplexed immunofluorescence staining. A five-color multiplex immunofluorescence panel based on tyramide signal amplification (TSA) was used for the simultaneous detection of CD3 (T cells), CD8 (cytotoxic T lymphocytes), Foxp3 (regulatory T cells), Ki67 (proliferating cells) and DAPI on tumor sections from formalin-fixed paraffin-embedded (FFPE) samples. The validation pipeline for the multiplex immunofluorescence protocol has been previously described (Sanchez-Paulete A et al., 2018). Briefly, 4-μm thick sections obtained from FFPE tissue blocks were deparaffinized and rehydrated from ethanol to water. Antigen retrieval with citrate (pH6, PerkinElmer) or EDTA (pH9, Dako) target retrieval solution was performed at the beginning of each sequential round of antibody staining. Each round consisted of heat induced antigen retrieval followed by protein blocking (Antibody Diluent/Block, Akoya Bioscience), incubation with primary antibody, anti-rabbit secondary antibody (Opal Polymer anti-rabbit HRP Kit, Perkin Elmer) finishing with Opal fluorophore incubation diluted in 1×Plus Amplification Diluent (Akoya Bioscience). The panel included the following primary antibodies: CD3 (rabbit monoclonal, clone SP7, 1:100, Abcam, ab16669), CD8 (rabbit monoclonal, clone D4W2Z, 1:500, Cell Signaling Technology, no. 98941), Foxp3 (rabbit monoclonal, clone D6O8R, 1:500, Cell Signaling Technology, no. 12653) and Ki67 (rabbit polyclonal, 1:500, Abcam, ab15580). At the end of the protocol, nuclei were counterstained with spectral DAPI (Akoya Biosciences) and sections were mounted with Faramount Aqueous Mounting Medium (Dako). Whole tissue sections were scanned on a Vectra-Polaris Automated Quantitative Pathology Imaging System (Akoya Biosciences). Akoya Biosciences' Inform software (v2.4.8) was used to remove the autofluorescence determined by an unstained slide and to perform the spectral unmixing of the images. Informative fields were selected for microphotography.

Statistical Analysis. Each experiment was performed using five to six mice per group. A one-way ANOVA test with Tukey post-test analysis, a two-way ANOVA test with Tukey post-test analysis, and a Log-rank test were used when appropriate to determine statistical significance (Graphpad Prism 6, La Jolla, Calif., USA). P-values were considered statistically significant when p<0.05. Other reference, statistical values are indicated in the description of the Figures herein.

Results

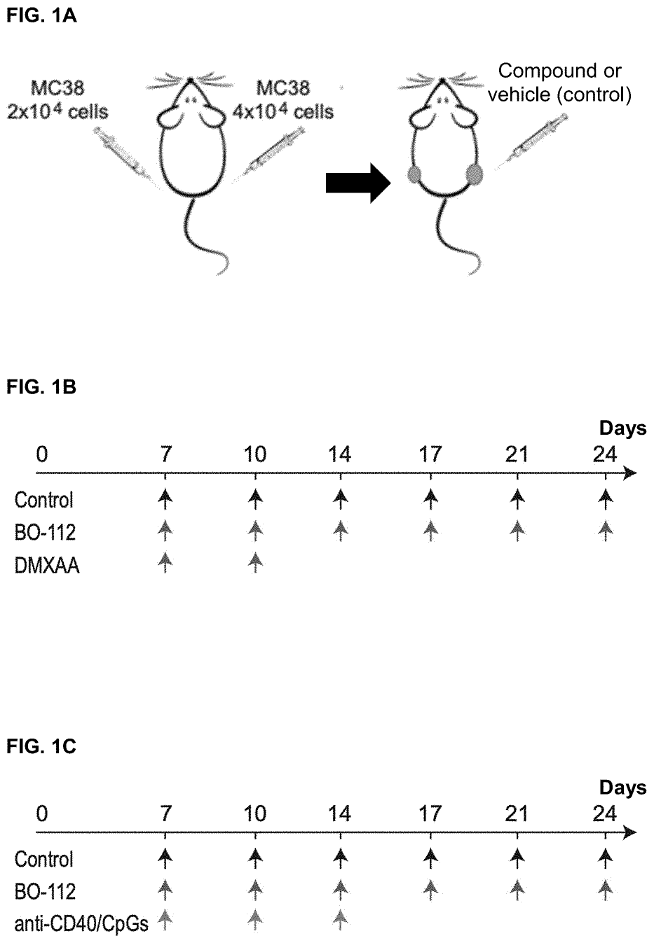

Distant efficacy following intratumoral immunotherapy is crucial to achieve clinically meaningful control of untreated tumor lesions, as well as long-term therapeutic effects. Therefore, a selection of immunotherapy agents is comparatively tested by means of repeated intratumoral injections in mice models bearing bilateral tumors having different origin (FIG. 1A, FIG. 1B, FIG. 1C). The tumor growth over time both in treated and untreated tumor lesions and survival is followed in parallel with the survival of mice in each control or test group. In single agent experiments, data essentially confirm published evidences, since directly injected tumors remarkably responded to the treatment with BO-112, DMXAA (as a reference STING agonist), ODN-1668 (as a reference class B CpG oligonucleotide), and ODN-2395 (as a reference class C CpG oligonucleotide), and not to anti-CD40 antibody or ODN-1585 (as a reference class A CpG oligonucleotide). In contrast, untreated contralateral tumors lethally progressed in most animals independently of the treatment, apart from few cases in which their growth is only delayed.

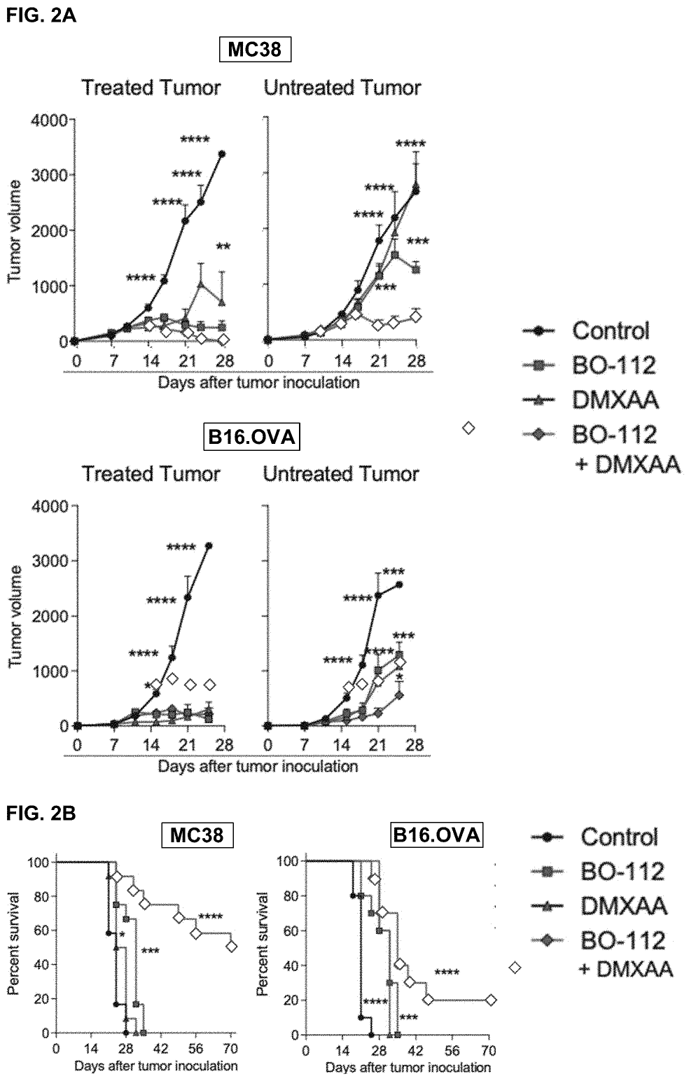

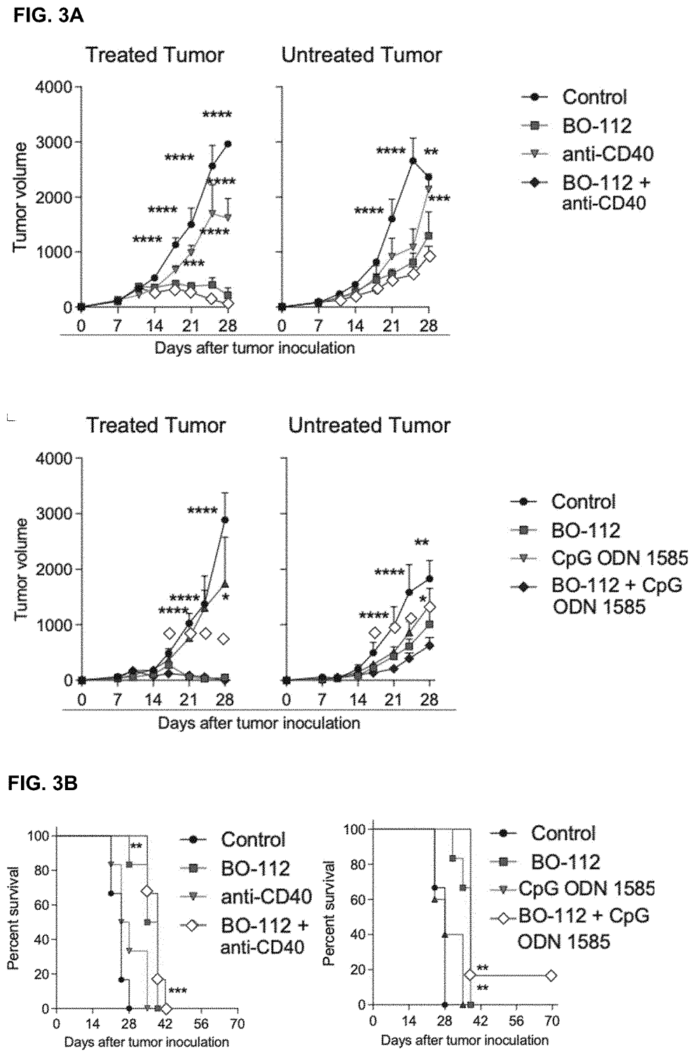

The potential improvement in the efficacy of intratumoral BO-112 administration in repressing untreated, distant metastatic disease is tested by co-injecting BO-112 with another agent in the same tumor mass. Such anti-metastatic effect is indeed observed only when DMXAA is injected with BO-112 in the same tumor mass that is originated from MC38 cancer cells or, to a lower but still significant extent, B16.OVA cancer cells (FIG. 2A). This intratumoral co-injection not only completely represses the growth of injected lesions but also eradicate distant disease in six out of twelve MC38-injected mice, when the intratumoral injection of only one compound achieves very limited effect on untreated lesions. Such local and distant combined efficacy effect on tumors leads to long-term survival (FIG. 2B). This effect on tumor growth and mice survival is not observed when the other compound agonistic anti-CD40 monoclonal antibody and class A/B/C CpG oligonucleotides (FIG. 3A, FIG. 3B), indicating that synergistic effects against untreated lesions clearly depend from the highly specific, unexpected effect of combining a STING agonist with BO-112 when intratumoral administration is performed for both agents. Clinically feasible co-injections of BO-11X and a STING agonist would then attain synergistic efficacy, in particular to eradicate distant untreated tumor lesions.

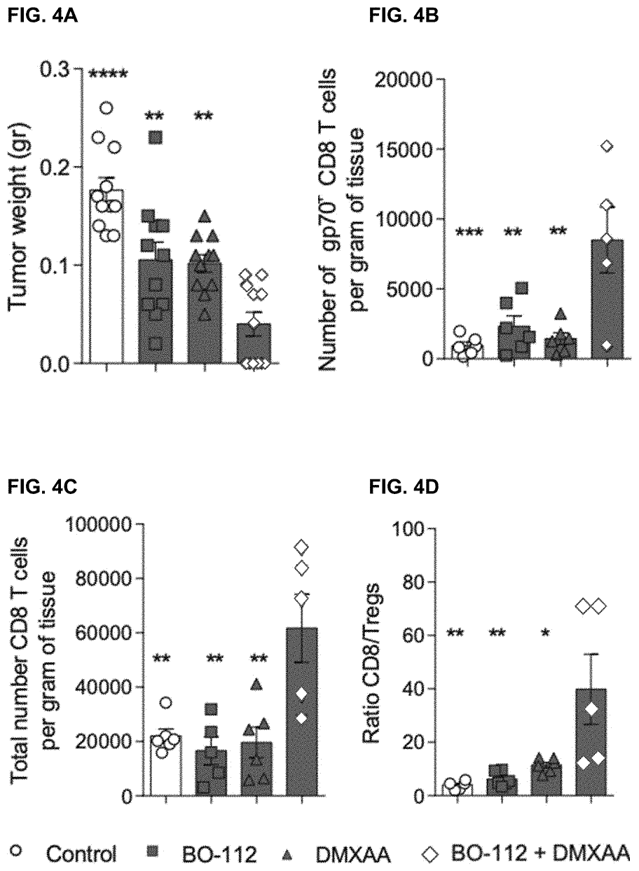

The mechanistic features explaining the synergistic effects of co-injections of BO-112 and STING agonists for local and systemic cancer treatment can be associated to the presence of anti-tumor CD8+ T-cell lymphocytes. The MC38-originated tumors from mice in the previously described control, single agent, or combined agent groups are surgically excised on day eleven (when suppressing effects on tumor size start to be macroscopically evident) and cell suspensions from such tumors are analyzed by multiparametric flow cytometry to find any association between intratumoral co-injections with stronger CD8 T cell-mediated anti-tumor effects. BO-112 and DMXAA-injected tumors have a much lower weight (FIG. 4A) and contain much more CD8 T cells specific for the gp70 immunodominant tumor epitope with H2-kb pentamers (FIG. 4B). Moreover, larger quantities of CD8 T cells per gram of tumor tissue are identified in BO-112 and DMXAA-injected tumors when compared to BO-112 or DMXAA single-agent injected tumors or tumors injected with vehicle (FIG. 4C). These synergistic, positive effects achieved following the intratumoral co-injection paradigms according to the present invention also result in higher CD8 to Treg ratios (FIG. 4D). In contrast, the numbers of conventional CD4 T cells (CD4⁺Foxp3⁻) within the tumor microenvironment were not altered by intratumoral co-injections of BO-112 and the STING agonist. These findings are further confirmed by evaluating the CD8 T cell and Treg component of BO-112 and DMXAA-injected tumors using multiplexed tissue immunofluorescence. Strikingly, four out of five cases of co-treated malignant tissues are completely necrotic and surrounded by an infiltrate in which neutrophils are prominent. From the point of view of circulating cytokines or chemokines, BO-112 resulted in a clear increase in type I Interferons, IL-12, IL-18, TNFalpha (TNFα) and CXCL10. However, the STING agonist either did not cause systemic circulating elevations or only enhanced those observed upon treatment with BO-112. These patterns may become important to avoid safety concerns due to excessive systemic inflammation or cytokine release syndromes.

The synergistic effects of intratumoral co-injection of BO-112 (the reference TLR3 agonist-containing particles suitable for intratumoral administration) and DMXAA (a reference STING agonist) can be additionally evaluated by injecting the two compounds in the same or two separate tumor masses using a different experimental approach where three tumor masses are established but only one or two are treated (FIG. 5A). Local and distant efficacy is indeed reproduced upon co-injections of BO-112 and DMXAA also when two different lesions are separately treated with BO-112 or DMXAA, efficacy against both treated tumors being preserved and in eight out of 12 mice and third-party untreated tumor lesions also completely regressing (FIG. 5B). Both approaches trigger a significant anti-tumor, systemic response also against untreated metastasis-like tumors.

Intratumoral immunotherapy has been thus far involving locoregional delivery of a single agent that may be administered with substances that facilitate pharmacokinetics and pharmacodynamics or protect and extend the action of the immunologically active compound. However, as shown in the experiments described herein, only specific immunotherapy agents may advantageously act by intratumoral co-injections. A poly(I:C)-based composition with agonistic effects on double-stranded RNA sensors such as BO-112 show significant abscopal, distant effects when combined with a STING agonist by intratumoral administration. Such beneficial effects have not been observed upon intratumoral co-injections of such poly(I:C)-based composition with other immunotherapy agents such as an anti-CD40 agonist monoclonal antibody or reference CpG-based oligonucleotides. Even if BO-112 and STING agonist may act on partially overlapping pathways, their combination for intratumoral injections can be used in methods and compositions able to control untreated distant disease in a synergistic manner.

### Example 2: Effects of Intratumoral Co-Injection of the Poly(I:C)-Based Composition BO-112 with a STING Agonist in Combination with Other Cancer Treatments

Materials & Methods

Evaluation of compounds. PD-1 blockade therapy was provided by intraperitoneal injection of anti-PD-1 antibody (clone RMP1-14, BioXcell; 100 μg) on days 8, 10, and 12. Control mice received intratumoral injections of PBS containing 5% glucose and/or 5% DMSO (vehicle), or intracellular injections of rat IgG. rIgG (30 μg). Cancer animal model and other experimental details are described in Example 1.

Results

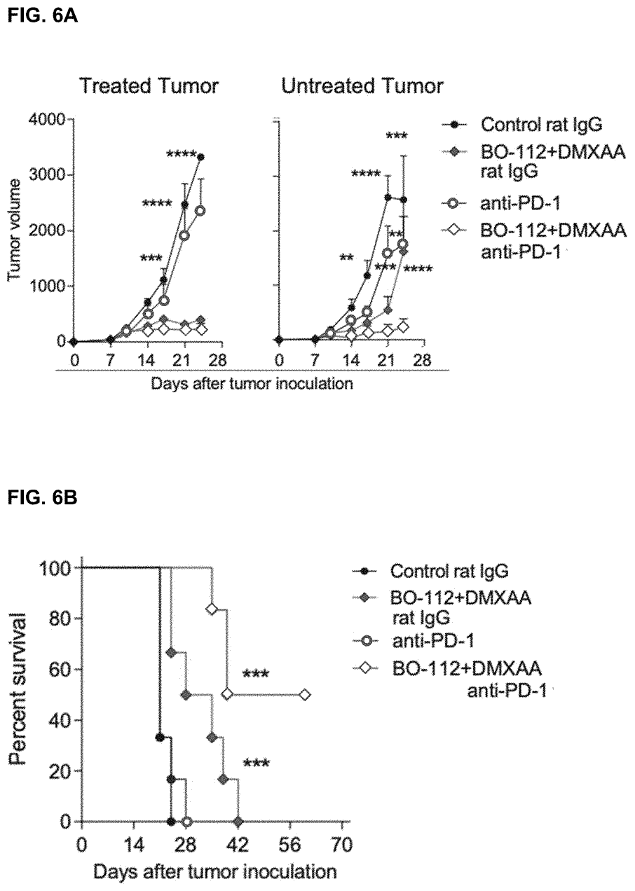

It is important to evaluate how the intratumoral co-injections of BO-112 (a reference TLR3 agonist-containing particles suitable for intratumoral administration) and DMXAA (a reference STING agonist) may be suitable and effective in the therapy of treated and untreated tumor lesions when included in combination therapies that can be administered using other routes and/or having a different nature or mechanism of action, such as radiotherapy or systemic administration of antibodies targeting cancer-relevant antigens. One example is the combination of such agents with an antibody that blocks PD-1, an approach that is now considered as a backbone in immunotherapy drug combinations (Ribas A and Wolchok J, 2018; Sharma P et al., 2021). This hypothesis can be tested in the same model presented in Example 1 (B16.OVA bilateral tumors) by including a systemic PD-1 blockade via intraperitoneal injections of an anti-PD-1 monoclonal antibody. The intratumoral co-administration of BO-112 and DMXAA clearly enhances the efficacy against non-directly injected tumors, with half of the mice receiving this drug regimen that rejects both treated and untreated tumors (but with specific, major improvements observed in the latter case), resulting in a clear increase of overall survival (FIG. 6A, FIG. 6B).

Thus, the intratumoral administration of BO-112 a reference TLR3 agonist comprising poly(I:C) molecules that is formulated for efficient intratumoral administration, and DMXAA, a reference STING agonist, was shown to provide a specific, synergistic therapeutic effect surprisingly also against untreated tumors (thus mimicking metastasis). The two immunotherapy agents can be delivered to the same, accessible tumor (in separate injections but possibly also when mixed in a single formulation including both agents to be administered using a single syringe) or in separate, accessible tumor lesions. These findings on the co-formulation efficacy against contralateral tumors that are refractory to either single-agent therapy suggest a number of clear advantages for clinical applications. For instance, this intratumorally-administered drug combination may be included in therapeutic protocols that are based on checkpoint inhibitors (e.g., systemic PD-1 blockade by means of anti-PD-1 or anti-PD-L1 antibodies) or radiotherapy (e.g., local low-dose radiotherapy), as well as in clinical proof-of-concept studies involving the administration of cancer antigens (cancer vaccination) or virotherapy (oncolytic viruses), even in an early stage of solid tumors (Hong W X et al., 2020). The dosing amounts and schedules of the poly(I:C)-based compositions with agonistic effects on double-stranded RNA sensors including TLR3 or MDA5 (e.g. preferably BO-112 or alternative formulations defined as BO-11X; further TLR3 agonists in cancer therapy are described in Le Naour J et al., 2020a) and a STING agonist (e.g. DMXAA or other compound acting as an agonist on human STING under clinical development; Motedayen Aval L et al., 2020; Flood B et al., 2019; Le Naour J et al., 2020b) may be adapted to the type and status of the subject cancer, as well as to type and the intensity of the relevant immune response (e.g. CD8 T cell response or cytokine-mediated responses) that provide therapeutic results in treated and untreated, distant tumors or metastasis due to abscopal effects.

Recent clinical studies using intratumorally injected drugs together with advancements in needles, biomaterials, and micro-/nanotechnologies (Muñoz N et al., 2021; Abdou P et al., 2020; Huang A et al., 2020; Subbotin V and Fiksel G, 2019) may additionally allow optimizing the therapeutic approaches according to the present invention for enhancing efficacy while minimizing toxicity, discomfort and/or complexity of intratumoral injections, triggering local and systemic immunologic responses of clinical interest (Hamid O et al., 2020). In addition, the site or sites of the tumor lesion(s) used for intratumoral injection may also evaluated to identify those cell types that are internalizing the BO-11XL formulations (WO 2020/104628) to thereby confer a clinical benefit, when combined with the intratumoral administration of a STING agonist, by decreasing not only the size of the injected tumor lesion, but also its metastatic potential that is observed at a distant, non-injected location (e.g. a subcutaneous nodule, a lymph node a skin lesion, or other lesion located in a visceral organ such as liver or lung). This potentially differential effect may require further evaluation which can comprise analysing one or more biopsies and also circulating cells such as dendritic cells, macrophages and other cells involved in immune responses in addition to proteins including chemokines, interferons, and cytokines in general, within a blood sample derived from a subject.

### CITED REFERENCES

Abdou P et al., 2020. Wiley Interdiscip Rev Nanomed Nanobiot 12:e1632 (PMID: 32255276)
Ager C et al., 2017. Cancer Immunol Res. 5:676-684 (PMID: 28674082)
Alvarez, M. et al. 2019. JCI Insight 5: e127729 (PMID: 31211693).
Alvarez M. et al., 2020. Front Immunol 11:7 (PMID: 32117218).
Aznar M et al., 2017. J Immunol 198:31-9 (PMID: 27994166)
Aznar M et al., 2019. J lmmunother Cancer 7:116 (PMID: 31046839)
Chelvanambi M et al., 2021. J lmmunother Cancer 9:e001906 (PMID: 33526609)
Corbett T et al., 1975. Cancer Res 35:2434-9. (PMID: 1149045)
Corrales L et al., 2015. Cell Rep. 11:1018-30 (PMID: 25959818)
Flood B et al., 2019. Immunol Rev. 290:24-38 (PMID: 31355488)
Hamid O et al., 2020. Oncologist. 25: e423-e438 (PMID: 32162802)
Harrington K et al., 2018. Annals of Oncology 29, Suppl. 8: 712 (Ann Oncol)
Hong W X et al., 2020. Clin Cancer Res. 26: 3091-3099. (PMID: 32071116)
Huang A et al., 2020 J Control Release 326:203-221 (PMID: 32673633)
Kyi C et al., 2018. Clin Cancer Res 24:4937-4948 (PMID: 29950349)
Lang D et al 2020. J Clin Med. 9: 3483. (PMID: 33126715)
Le NaourJ et al., 2020a. Oncoimmunology 9:1771143 (PMID: 32934877)
Le NaourJ et al., 2020b. Oncoimmunology 9:1777624 (PMID: 32934881)
Liberini V et al, 2021. Molecules. 226:2201. (PMID: 33920423)
Linardakis E et al., 2002. Cancer Res 62:5495-50 (PMID: 12359759)
Marabelle A et al., 2017. Ann Oncol 28:xii33-xii43 (PMID: 29253115)
Marquez-Rodas I et al., 2020. Sci Transl Med 12:eabb0391 (PMID: 33055241)
Motedayen Aval L et al., 2020. J Clin Med 9:3323 (PMID: 33081170)
Munoz N et al., 2021. J Immunother Cancer 9:e001800 (PMID: 33589526)
Pogue B et al., 2018. J Biomed Opt 23:1-9 (PMID: 30291698)
Ribas A et al., 2018. Cancer Discov 8:1250-7 (PMID: 30154193)
Ribas A and Wolchok J, 2018. Science 359:1350-1355 (PMID: 29567705)
Rodriguez-Ruiz M et al., 2018. Ann Oncol 29:1312-9 (PMID: 29554212)
Sanchez-Paulete A et al., 2018. Cancer research 78:6643-54 (PMID: 30297531)
Sharma P et al., 2021. Cancer Discov 11, 838-857 (PMID: 33811120).
Sivick K et al., 2018. Cell Rep 25:3074-3085 e5 (PMID: 30540940)
Subbotin V and Fiksel G, 2019. Am J Cancer Res 9:2209-2215 (PMID: 31720083)
Tselikas L et al., 2021. Clin Cancer Res 27: 1-8 (PMID: 33419781)
van de Donk P et al., 2020. Theranostics. 10: 1708-1718. (PMID: 32042331)
Xiong H et al., 2021. Int J Mol Sci. 22: 3295 (PMID 33804856)
Zahavi D and Weiner L, 2020. Antibodies (Basel) 9:34. (PMID: 32698317)
Zandberg D et al., 2020. Annals of Oncology 31, Suppl. 7:1446-1447 (AnnOncol)

1. A method for treating cancer comprising administering a stimulator of interferon genes (STING) agonist and a Toll-like Receptor 3 (TLR3) agonist to a subject in need thereof, wherein the STING agonist and the TLR3 agonist are administered intratumorally.

2. The method of claim 1, wherein the TLR3 agonist is a composition comprising polyinosinic-polycytidylic acid poly(I:C))) molecules that are complexed in one or more particles with polyethyleneimine (PEI).

3. The method of claim 1, wherein the STING agonist is a cyclic dinucleotide, a xanthenone, a flavonoid or a derivative thereof.

4. The method of claim 1, wherein the STING agonist and the TLR3 agonist are separately administered.

5. The method of claim 4, wherein the STING agonist and the TLR3 agonist are administered simultaneously or sequentially.

6. The method of claim 1, wherein the STING agonist and the TLR3 agonist are administered as a single composition.

7. The method of claim 1, wherein the STING agonist and the TLR3 agonist are administered in in two, three, or more cycles of treatment.

8. The method of claim 1, wherein a further drug or treatment is simultaneously or sequentially administered.

9. The method of claim 8, wherein the further drug or treatment is administered intratumorally, orally, subcutaneously, intradermally, intranasally, intravenously, intramuscularly, intrathecally, intranasally, intravesically, topically, or transdermally.

10. The method of claim 8, wherein further drug or treatment is surgery, radiotherapy, chemotherapy, toxin therapy, cancer vaccination, laser therapy, phototherapy, immunotherapy, cryotherapy or gene therapy.

11. The method of claim 10, wherein the further drug is an anti-PD-1 antibody or an anti-PD-L1 antibody.

12. The method of claim 1, wherein the cancer is melanoma, cervical cancer, liver cancer, breast cancer, ovarian cancer, pancreatic cancer, kidney cancer, prostate cancer, testicular cancer, urothelial carcinoma, bladder cancer, gastric cancer, non-small/small cell lung cancer, soft tissue sarcoma, colorectal adenocarcinoma, gastrointestinal stromal tumors, biliary tract cancer, gastroesophageal carcinoma, colorectal cancer, mesothelioma, myelodysplasia syndrome, transitional cell carcinoma, neuroblastoma, Wilms tumor, brain cancer, colon cancer, head/neck squamous cell carcinoma, hepatocellular carcinoma, or malignant melanoma.

13. The method of claim 12, wherein the cancer is metastatic.

14. The method of claim 12, wherein the cancer is refractory or resistant to radiotherapy, chemotherapy or immunotherapy.

15. A composition comprising a STING agonist and a TLR3 agonist and further comprising poly(I:C) complexed in one or more particles with PEI.

16. The composition of claim 15, wherein the TLR3 agonist is BO-112.

17. The composition of claim 15, wherein the TLR3 agonist is complexed with at least one STING agonist.

18. The composition of claim 15, wherein the composition is administered intratumorally.

19. A kit comprising the composition of claim 15 for intratumoral administration.

20. The kit of claim 19 further comprising one or more of diluents, adjuvants, excipients, syringes or other devices, and/or instructions for administration.
